(19) United States
(12) Patent Application Publication
(10) Pub. No.: US 2013/0115183 A1
(43) Pub. Date: May 9, 2013

(54) METHOD OF PREPARING HAIR AND SCALP CONDITIONING COMPOSITION INCLUDING AROMATIC OIL BLEND WITH ENHANCED EFFICACY

(71) Applicant: Do Yoon Ko, Seoul (KR)

(72) Inventor: Do Yoon Ko, Seoul (KR)

(21) Appl. No.: 13/805,714

(22) PCT Filed: Jun. 29, 2011
(86) PCT No.: PCT/KR11/04746

§ 371 Date: Jan. 22, 2013

(30) Foreign Application Priority Data
Jul. 1, 2010 (KR) 10-2010-0063280

(51) Int. Cl. A61K 8/97; A61Q 5/12; A61Q 5/02; A61Q 5/00

(52) U.S. Cl. CPC A61K 8/97; A61Q 5/00; A61Q 5/12; A61Q 5/006; A61Q 5/02
USPC 424/70.11; 424/74; 424/70.22; 424/70.31; 424/70.27; 424/70.21; 424/742

(57) ABSTRACT

Disclosed is a method of preparing a hair and scalp conditioning composition. According to the method, a hair cosmetic base and aromatic oils are aged by separate reaction processes and are blended to improve the stability of the aromatic oils. Therefore, the activities of the aromatic oils, particularly for the prevention of hair loss and dandruff, are enhanced.

### TECHNICAL FIELD

The present invention relates to a method of preparing a hair and scalp conditioning composition in which a hair cosmetic base and aromatic oils are aged by separate reaction processes and are blended to improve the stability of the aromatic oils, thereby achieving enhanced activities, particularly, effective prevention of hair loss and dandruff.

### BACKGROUND ART

Normal humans have about 100 to about 150 thousand hairs on the scalp. Each hair undergoes a growth cycle consisting of anaphase, metaphase, and telophase, and is finally shed. The period of this cycle lasts for 3-6 years. Humans lose an average of 60-100 hairs daily. In an alopecia patient, the proportion of hairs in the anaphase decreases and the proportions of hairs in the metaphase and telophase increase, resulting in abnormal hair loss.

Many hypotheses, for example, poor blood circulation, excessive functioning of male sex hormones, sebum hypersecretion, scalp dysfunction due to the presence of dandruff bacteria and other bacteria, hereditary factors, aging and stress are known as causes of alopecia. Complex functioning of these hypotheses causes various abnormal scalp and hair conditions as well as hair loss. Such abnormal scalp and hair conditions include poor hair restoration, hair growth and pilatory effects, sebum overproduction, dandruff bacteria proliferation, hair itch, scalp and hair dryness, poor hair flexibility and hair splitting. The hair growth refers to an effect that allows young hairs to grow well. Hairs become thicker and stronger due to the pilatory effect.

In recent years, people suffering from abnormal scalp and hair conditions are steadily on the rise due to changes in dietary habits, increased stress arising from complex social environments, etc. Further, the age of alopecia patients is dropping and the number of female patients with alopecia is increasing. Under these circumstances, research has been conducted on compositions that can prevent various abnormal scalp and hair conditions, including hair loss, and improve the conditions of scalp and hair.

Most conventional scalp and hair related products include chemical substances as major components. Accordingly, in many cases, long-term use of conventional products causes side effects, such as scalp stimulation.

Numerous attempts have been made to apply natural aromatic oils to scalp and hair related products in order to produce shampoos with hair loss preventive effects and hair growth efficacy.

Natural aromatic oils refer to natural essential oils with aromatherapeutic effects. Specifically, natural aromatic oils are highly concentrated vital oils extracted from flowers, leaves, stems, fruits, roots and other parts of medicinal herbs. About 70 kinds of natural aromatic oils can be used in humans. These natural aromatic oils may be used individually or as a mixture of two or three kinds thereof depending on intended purposes. All plants have the ability to proliferate and grow. Another ability of plants is to cure illness and heal injury. Natural aromatic oils take advantage of vital energy and curative power of plants.

Aromatic oils contain peculiar fragrance particles and enter the body through the nose and the skin to exert their pharmacological effects upon the body. This mechanism will be discussed briefly. First, fragrance particles are absorbed through the nose, which is a faster path than the skin. Small fragrance particles are inhaled into the nose through air as a medium and reach the brain. The fragrance particles deliver different stimuli to the brain depending on the shape thereof. The stimulated brain produces neurochemical substances possessing various effects, including relaxation, tension relief, stimulation and euphoria. These neurochemical substances affect the pituitary gland, where the secretion of hormones is regulated to exert pharmacological effects to the body. Fragrance particles may enter the body through the skin. In this case, fragrance particles are primarily absorbed through the hair follicles and the sweat glands and are then dissolved in lipids. The fragrance particles penetrate through the skin cells and into the dermis, circulate throughout the body along the capillaries and lymph glands, reach a certain organ having an affinity therefor, and stay in the organ. The fragrance particles exhibit their pharmacological effects while staying in the organ. Fragrance particles that exist in aromatic oils and exhibit their therapeutic effects are highly volatile and are readily dissolved in fats or oils. Such fragrance particles are complexes consisting of a number of ingredients and produce sweet smells. Fragrance particles are sensitive to external environments and tend to change in quality, color or smell.

In most case, conventional aromatic oil-containing cosmetics are produced by simply blending aromatic oils with general cosmetic base components in a single container. Therefore, it is difficult to minutely control temperature change during production processing, resulting in volatilization of fragrance ingredients of the aromatic oils or destruction of effective ingredients of the oils. Further, it is impossible to optimize the physical properties of the aromatic oils sensitive to external environmental conditions, resulting in deterioration of the stability of the oils. As a result, ingredients of the oils are not homogeneously blended with the cosmetic base components or are changed in color, quality and smell, resulting in deterioration of their activities.

### SUMMARY

### Technical Problem

The present invention has been made in an effort to solve the above problems, and it is an object of the present invention to provide a method of preparing a hair and scalp conditioning composition by aging a hair cosmetic base and aromatic oils by separate reaction processes and blending them to maintain the stability of the aromatic oils so that the activities of the aromatic oils are enhanced, achieving improved hair loss prevention, hair restoration, dandruff prevention and scalp irritation relief effects.

It is another object of the present invention to provide a hair and scalp conditioning composition prepared by the method.

### Technical Solution

According to an aspect of the present invention, there is provided a method of preparing a hair and scalp conditioning composition including: (a) dissolving a hair cosmetic base; (b) transferring and aging the hair cosmetic base; (c) blending two or more aromatic oils selected from the group consisting of lavender oil, rosemary oil, cedarwood oil, thyme oil, marjoram oil and eucalyptus oil, and aging the aromatic oil blend, separately from step (b); and (d) blending the aged hair cosmetic base of step (b) with the aged aromatic oil blend of step (c).

Step (a) is preferably carried out at 60 to 80° C. Steps (b), (c) and (d) are preferably carried out at 15 to 30° C., more preferably 18 to 23° C. The aging in each of steps (b) and (c) is preferably performed for 20 to 40 hours, more preferably 24 hours.

In step (c), the aromatic oils may be further blended with one or more oils selected from the group consisting of jojoba oil, argan oil, rosehip oil, sweet almond oil, olive oil, green tea seed oil, evening primrose oil, camellia oil, rice bran oil, safflower oil, apricot seed oil, avocado oil, cherry seed oil, grape seed oil, peanut oil, sunflower seed oil, hemp seed oil and wheat germ oil.

Preferably, the aromatic oil blend in step (c) includes lavender oil, rosemary oil, cedarwood oil, thyme oil, marjoram oil, eucalyptus oil and jojoba oil. In this case, lavender oil, rosemary oil, cedarwood oil, thyme oil, marjoram oil, eucalyptus oil and jojoba oil are preferably included in a weight ratio of 20-35:20-35:15-25:16-24:1-5:1-5:3-10, more preferably 25-30:25-30:17-20:18-21:1-3:1-3:5-8.

According to another aspect of the present invention, there is provided a hair and scalp conditioning composition prepared by the method.

The hair and scalp conditioning composition of the present invention is effective in hair loss prevention, hair restoration, dandruff prevention and scalp irritation relief due to enhanced activity of each of the aromatic oils.

### Advantageous Effects

The hair and scalp conditioning composition prepared by the method of the present invention exhibits improved hair loss prevention, hair restoration, dandruff prevention and scalp irritation relief effects due to enhanced activities of the aromatic oils. In addition, the hair and scalp conditioning composition uses reduced amounts of chemical additives, advantageously minimizing skin irritation.

### DETAILED DESCRIPTION

The present invention provides a method of preparing a hair and scalp conditioning composition including: (a) dissolving a hair cosmetic base; (b) transferring and aging the hair cosmetic base; (c) blending two or more aromatic oils selected from the group consisting of lavender oil, rosemary oil, cedarwood oil, thyme oil, marjoram oil and eucalyptus oil, and aging the aromatic oil blend, separately from step (b); and (d) blending the aged hair cosmetic base of step (b) with the aged aromatic oil blend of step (c).

Hereinafter, the constitutions of the method according to the present invention will be described based on the individual steps.

(a) Dissolution of Hair Cosmetic Base

In this step, a hair cosmetic base is dissolved with stirring in a container. The hair cosmetic base may be any of those commonly used in the art. The dissolution temperature is not particularly limited so long as the cosmetic base can be completely dissolved. The dissolution temperature is preferably from 60 to 80° C., more preferably 70° C. Too low a temperature makes it difficult to completely dissolve the base component. Meanwhile, too high a temperature may cause a change in the physical properties of components constituting the base or may increase the probability of volatilization of the base components.

The hair cosmetic base used in the present invention includes basic base components that are commonly used for the production of hair cosmetic formations. Such hair cosmetic formulations may be emulsions, creams, pastes, gels, cosmetic water, packs, lotions, powders, sprays and soaps. Specific examples of the hair cosmetic formulations include, but are not limited to, shampoos, rinses, hair packs, hair treatments, hair soaps, hair toners, hair lotions, hair creams, hair sprays, hair mousses and hair gels. Shampoos and rinses are preferred.

Specific examples of cosmetic base components constituting such formulations include dissolution aids, surfactants, moisturizers, thickeners, pH-adjusting agents, antioxidants, sequestering agents, colorants and flavors.

Examples of dissolution aids suitable for use as the base components include isopropyl myristate, polyethylene glycol, medium-chain fatty acid triglycerides, hydrocarbons and glycols.

The surfactants may be cationic polymers, anionic surfactants, nonionic surfactants, cationic surfactants and amphoteric surfactants. The cationic polymers function to maximize hair conditioning effects, and specific examples thereof include anionic polymers, such as polyquaternium-7, polyquaternium-10, polyquaternium-11 and polyquaternium-46. As the anionic surfactants, there may be used, for example, sodium lauryl sulfate, ammonium lauryl sulfate, magnesium lauryl sulfate, triethanolamine lauryl sulfate, sodium polyoxyethylene lauryl sulfate, ammonium polyoxyethylene lauryl sulfate and sodium laureth sulfate. As the nonionic surfactants, there may be used, for example, lauryl dimethylamine oxide, coconut oil alkyl dimethylamine oxide, lauric acid diethanolamide, coconut oil fatty acid diethanolamide, coconut oil fatty acid monoethanolamide, polyoxyethylene lauryl ether and polyoxyethylene nonyl phenyl ether. As the cationic surfactants, there may be used, for example, higher amine halides, quaternary ammonium salts and alkyl pyridinium salts. As the amphoteric surfactants, there may be used, for example, amino acids.

The moisturizers may be, for example, glycerin, propylene glycol, 1,3-butylene glycol, dipropylene glycol and sorbitol. The thickeners may be water soluble polymeric compounds, for example, methyl cellulose, hydroxyethyl cellulose, carrageenan, carboxymethyl cellulose and hydroxymethyl cellulose. Specific examples of the pH-adjusting agents include citric acid, sodium hydroxide, triethanolamine, sodium citrate, phosphoric acid, sodium phosphate and lactic acid.

Specific examples of the antioxidants include dibutylhydroxytoluene and ascorbic acid. The sequestering agents may be, for example, disodium ethylenediaminetetraacetate and tetrasodium ethylenediaminetetraacetate. Specific examples of disinfectants suitable for use as the base components include chlorhexidine gluconate, quaternary ammonium salts, piroctone olamine, pyrithione zinc suspension, iodopropynyl butylcarbamate and salicylic acid.

The cosmetic formulation may be a solution or an emulsion. In this case, the cosmetic formation may use a solvent, a solvating agent or an emulsifying agent as a base component. For example, water, ethanol, isopropanol, ethyl carbonate, ethyl acetate, benzyl alcohol, benzyl benzoate, propylene glycol, 1,3-butyl glycol oil, glycerol aliphatic ester, polyethylene glycol and fatty acid ester of sorbitan. In the case where the cosmetic formulation is a suspension, a carrier may be used. For example, the carrier may be selected from liquid diluents (such as water, ethanol and propylene glycol), suspending agents (such as ethoxylated isostearyl alcohol, polyoxyethylene sorbitol ester and polyoxyethylene sorbitan ester), microcrystalline cellulose, aluminum methahydroxide, bentonite, agar, and tragacanth.

The cosmetic formation may be a paste, a cream or a gel. In this case, animal fibers, plant fibers, waxes, paraffin, starch, tragacanth, cellulose derivatives, polyethylene glycol, silicon, bentonite, silica, talc and zinc oxide may be used as base components.

The cosmetic formation may be a powder or a spray. In this case, lactose, talc, silica, aluminum hydroxide, calcium silicate and polyamide powder may be used as base components. Particularly, the spray may further include a propellant, such as chlorofluorohydrocarbon, propane/butane and/or dimethyl ether.

The cosmetic base used in the method of the present invention is preferably a shampoo base. The shampoo base may contain shampoo base components known in the art. Examples of such shampoo base components, but are not limited to, cationic polymers, anionic surfactants, nonionic surfactants, cationic surfactants and amphoteric surfactants.

The hair cosmetic base is dissolved at 60 to 80° C. in a container with stirring until the components of the hair cosmetic base are completely dissolved. After dissolution, the base components may exist in a liquid or colloidal state. The stirring rate is from 20 to 30 rpm, preferably 24 rpm, when an agi mixer is used. The stirring rate is from 800 to 1,500 rpm, preferably 1,000 rpm, when a homo mixer is used. The dissolution temperature is from 60 to 80° C., preferably 70° C. When the cosmetic base is dissolved in the temperature range of 60 to 80° C., the volatilization of the base components can be minimized and the dispersion stability of the base solution increases.

In this step, the basic physical properties of the cosmetic base can be controlled by varying the dissolution factors, including temperature and time. The specific gravity and viscosity of the cosmetic base are maintained after dissolution. Therefore, the cosmetic base can be shaped into a desired formulation without the addition of any special additive, such as an emulsifying agent or a preservative. This advantageously minimizes the use of chemical components irritating the scalp.

After dissolution, the hair cosmetic base is preferably cooled to a temperature from 25 to 35° C., preferably 30° C. Particularly, in the case where the cosmetic base is a shampoo base, dissolution and cooling are preferably performed to adjust the pH, specific gravity and viscosity of the shampoo base to 5.5 to 6.0, 1.010 to 1.030 and 10,000 to 13,000 cps, respectively. By limiting the physical properties to the ranges defined above, the hair and scalp conditioning composition of the present invention can be shaped into a shampoo formulation without the use of any special chemical additive.

(b) Aging of the Hair Cosmetic Base

In this step, the hair cosmetic base dissolved in step (a) is transferred from the container to the outside, followed by aging.

Specifically, the hair cosmetic base is taken out of the container and transferred to another container, where it is aged. The aging is preferably performed at a temperature of 15 to 30° C., more preferably 18 to 23° C., for at least 20 hours, preferably 20 to 40 hours, more preferably 24 hours. The aging time may be varied depending on the kind of the cosmetic base. The aging of the hair cosmetic base under the temperature and time conditions defined above enables complete removal of the remaining heat of the cosmetic base component, increases the storage stability of the cosmetic base components, and does not damage the stability of aromatic oils during subsequent blending with the aromatic oils, which advantageously maintains the activity of effective ingredients of the aromatic oils without deterioration.

(c) Blending and Aging of Aromatic Oils

In this step, two or more aromatic oils are blended and aged, which are carried out separately from step (b). The aromatic oils are selected from the group consisting of lavender oil, rosemary oil, cedarwood oil, thyme oil, marjoram oil and eucalyptus oil.

The blending and aging are preferably performed at a temperature of 15 to 30° C., preferably 18 to 23° C., in a container separate from the container used in step (b). The aging time is at least 20 hours, preferably from 20 to 40 hours, more preferably 24 hours. The temperature range defined above acts as an equilibrium point where the stability of the aromatic oils can be maintained without deteriorating the activity of effective ingredients of the aromatic oils. The stabilization of the aromatic oils through the blending and aging in the temperature range defined above can maximize the activity of effective ingredients of the aromatic oils while minimizing the vaporization of fragrance from the aromatic oils during processing.

The aromatic oils used in the method of the present invention are essences extracted from natural plants. As the aromatic oils, there may be used, for example, lavender oil, rosemary oil, cedarwood oil, thyme oil, marjoram oil and eucalyptus oil. These oils may be optionally blended with one or more aromatic oils that exhibit hair and scalp conditioning efficacy. The optional aromatic oils increase the ability of the effective ingredients of the aromatic oils to be delivered to the skin to enhance the activities of the aromatic oils. As the optional aromatic oils, there may be used jojoba oil, argan oil, rosehip oil, sweet almond oil, olive oil, green tea seed oil, evening primrose oil, camellia oil, rice bran oil, safflower oil, apricot seed oil, avocado oil, cherry seed oil, grape seed oil, peanut oil, sunflower seed oil, hemp seed oil and wheat germ oil. Jojoba oil and argan oil are preferred.

Lavender oil assists in stress relief and deep sleep and is helpful to treat greasy scalp and alopecia scalp. Another efficacy of lavender oil is to promote the growth of cells and balance the secretion of sebum.

Rosemary oil is effective in inhibiting dandruff and preventing alopecia and has inflammation relief and astringent functions.

Cedarwood oil is effective against dermatitis, psoriasis and seborrheic alopecia.

Thyme oil has a preservative effect and antiseptic and sterilizing functions and is effective in preventing dandruff and hair loss.

Marjoram oil has sedative effects, relieves anxiety, nervous problems and insomnia, and is helpful for blood circulation.

Eucalyptus oil assists in oxygen delivery to increase skin respiration, cleans the fuzz out of the head, and is very effective against various inflammations.

Jojoba oil functions to deliver effective substances of the aromatic oil components to the skin, activate the scalp and supply nutrients.

Argan oil supplies moisture and nutrients to the skin to regenerate the skin and is effective in treating damaged, atopic and parched skin, eczema, etc.

Rosehip oil is effective to regenerate skin cells and strengthen cell membrane, maintains the moisture content of the skin and inhibits skin aging because it contains large amounts of linoleic acid and linolenic acid as essential fatty acids.

The aromatic oils used in the method of the present invention are commercially available. Essences extracted from the corresponding plants by techniques known in the art may be used as the aromatic oils.

The aromatic oils used in the method of the present invention may be selected from lavender oil, rosemary oil, cedarwood oil, thyme oil, marjoram oil, eucalyptus oil, and mixtures thereof. It is preferred to use all of these oils. Jojoba oil, argan oil or rosehip oil may be further added to and blended with the aromatic oil components.

Preferably, the aromatic oil blend includes lavender oil, rosemary oil, cedarwood oil, thyme oil, marjoram oil, eucalyptus oil and jojoba oil. In this case, lavender oil, rosemary oil, cedarwood oil, thyme oil, marjoram oil, eucalyptus oil and jojoba oil are include in a weight ratio of 20-35:20-35:15-25:16-24:1-5:1-5:3-10, more preferably 25-30:25-30:17-20:18-21:1-3:1-3:5-8. Within this weight ratio range, the aromatic oil blend creates a fresh and refreshed feeling and exhibits activities of the aromatic oils without skin irritation. Particularly, improved hair loss prevention, hair restoration, dandruff prevention and scalp irritation relief effects can be obtained simultaneously.

When the aromatic oil blend includes thyme oil, there is no need to add a preservative such as benzoic acid or paraoxybenzoic acid ester. Therefore, skin irritation arising from chemical substances can be minimized.

(d) Blending of the Hair Cosmetic Base and the Aromatic Oils

In this step, the aged hair cosmetic base of step (b) is blended with the aged aromatic oil blend of step (c) at 18 to 23° C. The aromatic oils are blended in an amount of 0.01 to 10 parts by weight, preferably 0.5 to 5 parts by weight, based on 100 parts by weight of the hair cosmetic base. The use of the aromatic oils in an amount of less than 0.01 parts by weight makes it difficult to expect the activities of the aromatic oils. Meanwhile, the use of the aromatic oil in an amount of more than 10 parts by weight may cause skin irritation.

The blending is preferably performed at a temperature of 18 to 23° C. Within this temperature range, the aromatic oils can be blended with the base components without deterioration of their stability.

After blending, the container is preferably filled in a product container at a temperature of 18 to 23° C., which is the same as the blending temperature, to maintain the stability of the oils.

The present invention also provides a hair and scalp conditioning composition prepared by the method.

The hair and scalp conditioning composition of the present invention is effective in hair loss prevention, hair restoration, dandruff prevention and scalp irritation relief due to enhanced activity of each of the aromatic oils.

### EXAMPLES

The constitutions and functions of the present invention will be explained in more detail with reference to the following preferred examples. However, these examples are provided to assist in a further understanding of the invention. Disclosures that are not included herein will be readily recognized and appreciated by those skilled in the art, and thus their description is omitted.

### Example 1

### Production of Aromatic Oil-Containing Shampoo Using Tank-Out Process (Separation Process)

(a) 1 kg of a shampoo base containing sodium laury sulfate, sodium laureth sulfate and polyquaternium-7 was completely dissolved with stirring at 1,000 rpm using a homo mixer in a container at 70° C. for 20 min.

(b) After dissolution, the shampoo base was transferred to the outside of the container, followed by aging at 20° C. for 24 hr.

(c) Next, 2.7 g of lavender oil, 2.7 g of rosemary oil, 1.8 g of cedarwood oil, 1.9 g of thyme oil, 0.1 g of marjoram oil, 0.1 g of eucalyptus oil and 0.7 g of jojoba oil were blended in another container at 20° C., followed by aging for 24 hr.

(d) 10 g of the aromatic oil blend was blended with 990 g of the aged hair cosmetic base at 20° C., and the blend was then filled in a shampoo container at the same temperature to produce an aromatic oil-containing shampoo.

### Example 2

### Production of Aromatic Oil-Containing Shampoo

A shampoo was produced in the same manner as in Example 1, except that steps (b), (c) and (d) were carried out at 10° C.

### Example 3

### Production of Aromatic Oil-Containing Shampoo

A shampoo was produced in the same manner as in Example 1, except that steps (b), (c) and (d) were carried out at 40° C.

### Comparative Example 1

### Production of Aromatic Oil-Containing Shampoo Using Tank-In Process (Integration Process)

900 g of a shampoo base containing sodium laury sulfate, sodium laureth sulfate and polyquaternium-7 was dissolved with stirring at 1,000 rpm using a homo mixer in a container at 70° C. Next, 2.7 g of lavender oil, 2.7 g of rosemary oil, 1.8 g of cedarwood oil, 1.9 g of thyme oil, 0.1 g of marjoram oil, 0.1 g of eucalyptus oil and 0.7 g of jojoba oil were blended at room temperature. The aromatic oil blend was directly added to and blended with the shampoo base dissolved in the container at 50-60° C. without undergoing aging of the shampoo base and aromatic oils. The resulting blend was filled in a shampoo container to produce an aromatic oil-containing shampoo.

### Comparative Example 2

### Commercially Available Shampoo

A commercially available aromatic oil-containing shampoo for hair loss prevention was prepared.

### Test Example 1

### Evaluation of Hair and Scalp Conditioning Effects

The shampoos produced in Examples 1-3 and Comparative Examples 1-2 were evaluated for hair loss prevention, hair growth and dandruff prevention efficacy. Specifically, 250 subjects suffering from or undergoing hair loss were divided into 5 groups (each 50 subjects). Each group was allowed to use the corresponding shampoo for a month (at least once daily). The efficacy parameters were evaluated using a 5-point scale and scored based on the following criteria: Excellent (5), good (4), average (3), little effect (2), no effect (1). The results are shown in Table 1.

1. Hair loss preventive effect

2. Hair thickening effect

3. Hair growth and pilatory effects

4. Dandruff and itch preventive effects

5. Seborrheic scalp improving effect

6. Detergency (Deep cleansing; Bubble size)

7. Fragrance preference

TABLE 1

| | Example | Example | Example | Comparative | Comparative |
|---|---|---|---|---|---|
| | 1 | 2 | 3 | Example 1 | Example 2 |
| Hair loss | 4.88 | 4.14 | 4.29 | 3.74 | 3.08 |
| preventive | | | | | |
| effect | | | | | |
| Hair | 4.92 | 4.03 | 4.19 | 3.67 | 2.8 |
| thickening | | | | | |
| effect | | | | | |
| Hair | 4.84 | 4.11 | 4.38 | 3.72 | 2.87 |
| growth and | | | | | |
| pilatory | | | | | |
| effects | | | | | |
| Dandruff | 4.93 | 4.28 | 4.41 | 3.59 | 2.52 |
| and itch | | | | | |
| preventive | | | | | |
| effects | | | | | |
| Seborrheic | 4.98 | 4.35 | 4.27 | 3.64 | 2.72 |
| scalp | | | | | |
| improving | | | | | |
| effect | | | | | |
| Detergency | 4.84 | 4.68 | 4.61 | 4.21 | 3.68 |
| Fragrance | 4.87 | 4.66 | 4.52 | 4.01 | 3.46 |
| preference | | | | | |

As can be seen from the results in Table 1, the inventive shampoo produced using a separation process in Example 1 was excellent in all parameters, including hair loss preventive effect, hair thickening effect, hair growth and pilatory effects, dandruff and itch preventive effects, seborrheic scalp improving effect, detergency and fragrance preference, in comparison with the shampoo produced by an integration process in Comparative Example 1 and the commercially available shampoo of Comparative Example 2.

In addition, the shampoo of Example 1, which was produced by blending the shampoo base with the aromatic oils and aging the blend at 20° C., showed excellent hair and scalp conditioning effects as a whole when compared to the shampoo of Example 2, which was produced by blending and aging at 10° C., and the shampoo of Example 3, which was produced by blending and aging at 40° C.

### INDUSTRIAL APPLICABILITY

The hair and scalp conditioning composition prepared by the method of the present invention exhibits hair loss prevention, hair restoration promotion, dandruff prevention and scalp irritation relief effects while minimizing skin irritation. Therefore, the hair and scalp conditioning composition can be useful in the production of hair preparations, for example, shampoos, rinses, treatments and hair lotions.

1. A method of preparing a hair and scalp conditioning composition, comprising:
(a) dissolving a hair cosmetic base;
(b) transferring and aging the hair cosmetic base;
(c) forming an aromatic oil blend by blending two or more aromatic oils selected from the group consisting of lavender oil, rosemary oil, cedarwood oil, thyme oil, marjoram oil and eucalyptus oil, and aging the aromatic oil blend, separately from step (b); and
(d) blending the aged hair cosmetic base of step (b) with the aged aromatic oil blend of step (c),
wherein steps (b), (c) and (d) are carried out at 15 to 30° C.

2. The method according to claim 1, wherein step (a) is carried out at 60 to 80° C.

3. The method according to claim 1, wherein steps (b), (c) and (d) are carried out at 18 to 23° C.

4. The method according to claim 1, wherein the aging in each of steps (b) and (c) is performed for 20 to 40 hours.

5. The method according to claim 1, wherein the hair cosmetic base is a base for a formulation selected from the group consisting of shampoos, rinses, hair packs, hair treatments, hair soaps, hair toners, hair lotions, hair creams, hair sprays, hair mousses and hair gels.

6. The method according to claim 1, wherein the hair cosmetic base is a shampoo base comprising at least one component selected from the group consisting of cationic polymers, anionic surfactants, nonionic surfactants, cationic surfactants and amphoteric surfactants.

7. The method according to claim 1, wherein in step (c), the aromatic oil components are further blended with one or more oil components selected from the group consisting of jojoba oil, argan oil, rosehip oil, sweet almond oil, olive oil, green tea seed oil, evening primrose oil, camellia oil, rice bran oil, safflower oil, apricot seed oil, avocado oil, cherry seed oil, grape seed oil, peanut oil, sunflower seed oil, hemp seed oil and wheat germ oil.

8. The method according to claim 7, wherein in step (c), the aro-matic oil blend comprises lavender oil, rosemary oil, cedarwood oil, thyme oil, mar-joram oil, eucalyptus oil and jojoba oil.

9. The method according to claim 8, wherein the lavender oil, rosemary oil, cedarwood oil, thyme oil, marjoram oil, eucalyptus oil and jojoba oil are present in a weight ratio of 20-35:20-35:15-25:16-24:1-5:1-5:3-10.

10. The method according to claim 1, wherein in step (d), the aro-matic oils are blended in an amount of 0.01 to 10 parts by weight, based on 100 parts by weight of the hair cosmetic base.

11. The method according to claim 6, wherein after dissolution in step (a), the pH, specific gravity and viscosity of the shampoo base are adjusted to 5.5 to 6.0, 1.010 to 1.030 and 10,000 to 13,000 cps, respectively.

12. A hair and scalp conditioning composition prepared by the method according to claim 1.

13. The composition according to claim 12, wherein the composi-tion is used for hair loss prevention, hair restoration, dandruff prevention or scalp irritation relief.
